(19) United States
(12) Patent Application Publication
(10) Pub. No.: US 2024/0358638 A1
(43) Pub. Date: Oct. 31, 2024

(54) FOAM COMPOSITIONS FOR TREATING CLOSTRIDIOIDES DIFFICILE INFECTIONS

(71) Applicant: TDL INNOVATIONS LLC, Princeton, NJ (US)

(72) Inventor: Glenn W. Laub, Princeton, NJ (US)

(21) Appl. No.: 18/556,444

(22) PCT Filed: Apr. 22, 2022
(86) PCT No.: PCT/US2022/025934

§ 371 Date: Oct. 20, 2023

Related U.S. Application Data
(60) Provisional application No. 63/178,234, filed on Apr. 22, 2021.

(51) Int. Cl. A61K 9/12; A61K 9/00; A61K 31/7048; A61K 38/14; A61K 47/06

(52) U.S. Cl. CPC A61K 9/122; A61K 9/0053; A61K 31/7048; A61K 38/14; A61K 47/06

(57) ABSTRACT

Disclosed is the use of foam compositions for the delivery of therapeutics to the colon of a patient in need thereof. Also disclosed are foam compositions, and devices for delivering the composition to the colon. Additionally, methods of treating C. difficile in the colon using the foam compositions are also disclosed.

### CROSS REFERENCE TO RELATED APPLICATIONS

This application claims the benefit of U.S. Provisional Application No. 63/178,234, filed Apr. 22, 2021, which is incorporated herein by reference in its entirety.

### FIELD

The present disclosure provides foam compositions for the treatment of gastrointestinal infection, for example by Clostridioides difficile, by the delivery of the foam composition containing one or more therapeutic agents to the rectum and/or colon of a patient in need thereof. Also disclosed are foam compositions, and devices for delivering the composition to the rectum and/or colon. Additionally, methods of treating gastrointestinal infections using the foam compositions are disclosed.

### BACKGROUND

The gastrointestinal (GI) tract is naturally inhabited by as many as one thousand different bacterial species that live symbiotically with their host. If this microbiota is disrupted, for example due to antibiotics or illness, invasion by toxic pathogens may lead to GI infections. GI infections may also be caused by ingestion of contaminated food and water and can be viral, bacterial or parasitic in nature. Symptoms include inflammation of the GI tract, diarrhea, vomiting, and abdominal pain as well as systemic toxicity, peritonitis, toxic colonic dilatation, and bowel perforation in more dire cases.

Clostridioides difficile infection (CDI) is a GI infection caused by the spore-forming, anaerobic, toxin-producing intestinal bacterium, Clostridioides difficile (C. difficile, or C. difficile). C. difficile is a strict anaerobe that is highly virulent due in part to its ability to produce spores. These spores are resistant to environmental insults such as heat and ethanol based disinfectants. When the spores are ingested, once they are able to reach the intestine and attach to the epithelial cells in order to germinate. Disturbance of a healthy gut microbiota allows germination and propagation of the infection, and subsequent release of symptom-causing toxins.

The predominant pathogenicity factors of C. difficile that are responsible for clinical symptoms are known as toxin A (TcdA) and toxin B (TcdB). These toxins are responsible for the inflammatory response as well as degradation of intestinal epithelial cells. Damage to the colonic mucosa eventually leads to diarrhea and shedding of spores which have the capability of infecting new hosts.

There are several risk factors known to cause CDI. These include exposure to C. difficile spores, advanced age, underlying disease, impaired immune response, as well as disruption of the intestinal flora and subsequent colonization with C. difficile. A significant cause for the disturbance of indigenous gut microbiota is the administration of certain antibiotics, which can disrupt the microbiota for several months post-treatment, increasing the susceptibility to CDI.

Currently, the treatment approach for CDI is dependent upon the severity and circumstances of the infection. The current primary treatment is the enteral administration of antibiotics. In addition, alternative therapies exist including probiotics, fecal microbiota transplantation, secondary bile acids such as ursodeoxycholic acid, and anti-sporulation therapeutics. In severe cases, for patients who are not responding to treatment, systemic toxicity, peritonitis, toxic colonic dilatation, and bowel perforation may develop leading to surgical intervention.

There is a marked risk of recurrent CDI and certain therapies focus on more persistent cases of the infection. Recurrences are due to failure of the immune system to respond to C. difficile as well as alteration to the normal protective microbiota. Disrupted intestinal flora may be replaced by fecal microbiota transplantation (FMT) obtained from healthy donors.

For most therapeutics to treat CDI, administration is generally oral, however certain populations may be difficult to treat with oral medicines, including children, the elderly and those with difficulty swallowing. An alternative and more direct method of treatment may be given rectally by way of suppositories or using an enema. However, administration of therapeutic agents directly to the intestine comes with significant limitations. Direct administration typically involves either suppositories or the use of a catheter to deliver a liquid enema containing the therapeutic agent to the affected area. Both of these methods have certain disadvantages. Suppositories have the disadvantage that they exhibit slow release and provide relatively little spreading throughout the rectum/colon, therefore the therapeutic agent may not be distributed widely enough to cover the target area completely. Delivery of the therapeutic agent in the form of a liquid enema via a catheter also has significant limitations. After delivery of the liquid containing the therapeutic agent to the targeted area, leakage tends to occur from the rectum or the liquid may accumulate at certain locations due to gravity causing uneven distribution of the therapeutic agent in the affected area. Not only does this lead to reduced efficacy of the treatment, the negative aspects of rectal leakage could also lead to low patient acceptance and compliance.

In view of the current treatment options, and the high mortality rates of CDI, there is an unmet need for a delivery mechanism where the GI infection therapeutic agent, in particular a CDI treatment, is effectively distributed to the target location, allowing higher local tissue concentration than systemic administration and causing minimal side effects. The present disclosure provides foam compositions for administration of one or more GI infection therapeutic agents, including CDI therapeutic agents, in a foam composition, which allows for effective distribution and improved retention of the therapeutic agent or agents in the affected area while minimizing side effects and patient discomfort.

### SUMMARY

In one aspect, the disclosure provides a method to treat an infection of the gastrointestinal (GI) tract in a patient in need thereof by administering a foam composition comprising a liquid component, a foaming agent, and further comprising one or more therapeutic agents. The gastrointestinal infection may be caused by bacteria, viruses, or parasites. In some embodiments, the gastrointestinal infection is caused by a bacteria such as Staphylococcus aureus, Bacillus cereus, Clostridium perfringens, Clostridium botulinum, Escherichia coli (ETEC, EPEC, EHEC, EAEC, EIEC), Salmonella sp., Shigella sp., Campylobacter sp., Yersinia enterocolitica, Clostridioides difficile, Vibrio cholerae, Vibrio parahemolyticus, Listeria monocytogenes, Aeromonas hydrophila, or Plesiomonas sp. In other embodiments, the gastrointestinal infection is caused by a parasite such as Entameba histolytica or Trichuris trichiura. In embodiments, the infection is a Clostridioides difficile infection. In some embodiments, the pathogen is antibiotic resistant.

In embodiments, the gastrointestinal infection may limited to the colon or it may be dispersed throughout the intestines. In some embodiments, the gastrointestinal infection is a primary infection. In some embodiments, the gastrointestinal infection is a recurrent infection. Additionally, the gastrointestinal infection may be asymptomatic.

In another embodiment, the disclosure provides a method to treat a gastrointestinal infection comprising administering a foam composition comprising a foaming agent and further comprising one or more therapeutic agents selected from anti-infective agents, antibiotic agents, anti-inflammatory agents, anti-sporulation agents, probiotic agents, anti-toxin antibodies, secondary bile acids, and fecal transplant.

In some aspects the therapeutic agent comprises one or more of vancomycin, metrolidazole, fidaxomicin, tigecycline penicillin, cephalosporin, antifolate/sulfa antibiotic combinations, nitroimidazole, penem, glycopeptide, and monobactam antibiotics.

In other aspects, the foam composition is sterile, i.e., free, or substantially free, of living microbes and/or bacteria. All ingredients of the composition may be sterilized separately, mixed, and packaged under aseptic conditions. Alternatively, the composition may be sterilized in its final form prior to administration. In other embodiments, the compositions comprise a pharmaceutically acceptable carrier and a cellulose derivative, which is heat sterilizable. Optionally, certain components may not be sterile.

The foamable compositions disclosed herein are capable of forming a foam before, during, or after administration of the foaming composition to the gastrointestinal tract, via the rectum. In addition to an active agent, e.g., a CDI treatment agent, the foam composition comprises a liquid component, one or more foaming agents (e.g., a surfactant or polymer) and one or more bubble-forming agents (e.g., a dissolved gas, or volatile liquid that forms a gas upon exposure to ambient temperature and/or body temperature and ambient pressure). The gas component may be a single gas or a mixture of gasses. The volatile liquid may be a fluorinated hydrocarbon (for example, H134a or FO1234ze).

In another aspect, the disclosure provides a device for delivery of the foam composition comprising one or more reservoirs for the therapeutic agent and propellant, a foam generator, regulator, actuator, and patient interface. The device may comprise one or more containers, e.g., two containers. The reservoir for the therapeutic agent may be one of the containers. The device may be a single use device or may be used multiple times.

In some embodiments, the device comprises two containers comprising (i) a foamable composition with one or more therapeutic agents and (ii) a pressurized gas canister. In other embodiments, the device comprises containers comprising (i) a foamable composition and (ii) one or more therapeutic agents and pressurized gas. In such embodiments comprising two containers, the compositions in the separate containers are combined prior to administering to the patient. Alternatively, the devices comprise a single container with (1) the liquid component, foaming agent, one or more therapeutic agents and propellant, or (2) the liquid component, foaming agent, one or more therapeutic agents, and dissolved gas. In another aspect, the therapeutic agent or propellant can be added to the device through a valve or port prior to administration. Additionally, the device will comprise a port or container outlet to attach a dispensing apparatus, e.g., catheter system or applicator nozzle.

### DETAILED DESCRIPTION

The present disclosure provides a composition for the delivery of one or more gastrointestinal (GI) infection therapeutic agents to the intestines of a patient in need thereof. Also disclosed are compositions and devices for delivering the composition to the intestines. Additionally, methods of treating GI infections using the foam compositions described herein are disclosed.

Therapeutics for treating GI infection are generally administered orally, however certain populations may be difficult to treat with oral medicines, including children, the elderly, those with difficulty swallowing, critically ill patients, patients who fail oral therapy, patients who have ileus, patients with altered GI motility, and patients who are nauseas. An alternative and more direct method of treatment may be given rectally by way of suppositories or using an enema. However, administration of therapeutic agents using such dosage forms comes with significant limitations. Suppositories have the disadvantage that they exhibit slow release and provide little spreading throughout the rectum/colon, therefore the therapeutic agent may not be distributed widely enough to cover the target area. Delivery of the therapeutic agent in the form of a liquid enema via a catheter also has significant limitations. After delivery of the liquid containing the therapeutic agent to the target area, leakage tends to occur from the rectum, or the liquid may accumulate at certain locations due to gravity causing uneven distribution of the therapeutic agent in the affected area. This leads to a reduced efficacy of the treatment, and the negative aspects of rectal leakage leads to low patient compliance.

Severe C. difficile infection (CDI) carries a high mortality rate. Early diagnosis and aggressive medical management is imperative to improve outcomes. Although enteral vancomycin is the primary recommended treatment for CDI, the presence of adynamic ileus may prevent oral vancomycin from reaching the distal colon. In patients with ileus, vancomycin retention enemas may be used to treat the distal colon adequately. However, although most guidelines recommend enemas of vancomycin in 100 mL for CDI treatment in patients with adynamic ileus, significant concern remains that a volume of 100 mL of 0.9% saline is not adequate to reach the distal or proximal colon. In view of this concern, it is important to review the evidence regarding the efficacy of vancomycin retention enemas in CDI.

In view of the current treatment options, there is an unmet need for a delivery mechanism where the GI infection therapeutic agent, in particular a CDI treatment, is effectively distributed to the target location, allowing higher local tissue concentration than systemic administration and causing minimal side effects. The present disclosure provides foam compositions for administration of one or more GI infection therapeutic agents, including CDI therapeutic agents, to a patient, which can allow effective distribution of the therapeutic agent or agents in the affected area while minimizing side effects.

Delivering a therapeutic agent directly to the intestines of a patient allows higher local tissue concentration of the GI infection therapeutic agent than systemic administration (e.g., by oral or i.v. route), which allows the therapeutic agent to be more effective while limiting the side effects. The foam compositions described herein effectively deliver the therapeutic agent directly to the intestines or part of the intestine in need of treatment. Systemic drug administration often requires high dosages or repeated administration to achieve a therapeutic effect, which increases the risk of side effects and lower patient tolerance. The present disclosure provides foam-based delivery systems containing a GI infection therapeutic agent that is delivered directly to the intestines or part of the intestine in need of treatment, thereby providing increased local tissue concentrations of the therapeutic agent than would be achieved by systemic administration. Additionally, administering the therapeutics agents in a foam composition can provide spatial and temporal control over the delivery and release of the therapeutic agents.

### Foam Compositions

The foam compositions contain at least one active ingredient in a biocompatible foamable carrier including but not limited to oleaginous foams, oil-in-water foams, water-in-oil foams, liposome-based foams, surfactant-based foams, gel-based foams and nanoparticle-based foams. As used herein a foam composition includes a foam composition, or a composition capable of forming a foam composition. In some embodiments, the foam comprises a liquid component, a foaming agent, and a bubble-forming agent, which are configured to form a foam. The foam composition further comprises a therapeutically effective amount of an active therapeutic agent (in particular a GI infection therapeutic agents) that may be dissolved, dispersed, or suspended in the liquid component. The foam compositions may further comprise one or more of a solvent, a stabilizer, and a preservative. In some embodiments, the composition may optionally comprise a foam adjuvant.

In one aspect of the disclosure, the composition is sterile (e.g., substantially free of microbes and/or bacteria). All ingredients of the composition are sterilized separately, mixed, and packaged under aseptic conditions. In other embodiments, the compositions comprise a pharmaceutically acceptable carrier and a cellulose derivative, which is heat sterilizable. In some embodiments, the composition is at least 80%, at least 90%, and up to 100% sterile. In other embodiments, components of the composition are not sterile.

In some embodiments, the foam formed by the foam composition is stable. In some embodiments, the foam retains at least 50%, at least 70%, at least 85%, at least 90%, at least 95%, or at least 99% of the original foam volume after two to four minutes of administration; after four to ten minutes of administration; or after ten to fifteen minutes of administration of the composition and foam formation. In some cases the foam persists for 1 to 2 hours, 1 to 4 hours, 1 to 6 hours, 1 to 8 hours, 2 to 4 hours, 2 to 6 hours, 2 to 8 hours, 2 to 10 hours, 3 to 5 hours, 4 to 8 hours, about 1 hour, about 2 hours, about 3 hours, about 4 hours, about 5 hours, about 6 hours, about 7 hours, about 8 hours, about 9 hours, or about 10 hours after the administration of the composition and foam formation.

In embodiments, the foam compositions include a liquid component, such as one or more of water, a non-aqueous solvent, and a hydrophobic organic carrier. The hydrophobic organic carrier may be propylene glycol. The compositions may comprise a solvent and/or an emulsifier, including but not limited to methyl paraben, propylparaben, and/or emulsifying wax. The compositions may optionally comprise a stabilizer, viscosity modifier, or bio-adhesive agent such as xanthan gum, locust bean, gum arabic, gum ghatti, guar gum, gum tragacanth, karaya gum, pullulan, alginate, carrageenan, pectin, gellan, chitosan, chondroitin sulfate, dermatin sulfate, and heparin.

The foam compositions also include a foaming agent that is present in the foam composition to provide structure and allowing bubbles to be trapped in the composition so that when a gas is evolved, a foam results. Thus, the foaming agent may be, for example, a polymer or a surfactant. The foam composition may contain one, two, three or more foaming agents. The foaming agent may be a surfactant including a biocompatible non-ionic surfactant (e.g., polysorbate 80, Laureth 23, or Poloxamer). In some other embodiments, the foaming agent may be a natural surfactant (e.g., albumin, gelatin, denatured collagen, soybean-derived proteins, and stearic acid).

In some embodiments, the composition may comprise one or more biocompatible surfactants. The one or more surfactants may include a biocompatible foaming agent selected to modify the stability of the foam formed after administering the composition to a patient. In some embodiments, the surfactants may further function as a sclerosing agent. Examples of such surfactants include phospholipids, neutral lipids, hydrophobic surfactants, biocompatible soaps or detergents, and combinations thereof. In some embodiments, the surfactant may be sodium tetradecyl sulfate, polysorbate 80, or poloxamer.

Poloxamers comprise co-polymers of a hydrophobic poloxypropylene (POP) moiety sandwiched between two hydrophilic moieties of polyoxyethylene (POE) and have the ability to solubilize lipophilic drugs within the hydrophobic core. Poloxamer based gel/foam formulations exhibit thermosensitive rheological properties, which may be advantageous for localized, sustained delivery of drugs. The foam composition may comprise one or more Poloxamer polymers selected from the group consisting of Poloxamer 407 (F127), Poloxamer 338 (F108), Poloxamer 188 (F68).

In some embodiments, the composition can be in the form of a thermo-sensitive gel. In some embodiments, the viscosity of the composition increases above a specific temperature, leading to a decreased diffusion of any therapeutic agents present in the composition leading to sustained delivery and prolonged localized “depot” in the targeted area of treatment. In embodiments where the foaming agent is a thermosensitive polymer or gelling agent, the viscosity of composition increases during or after administering the composition to the colon or rectum. In some embodiments, the foam composition comprises a gel or a composition which forms a gel, that is substantially liquid (low viscosity) at about room temperature (e.g., about 20° C. to about 25° C.), but as the temperature of the composition increases, the viscosity of the composition increases. In some embodiments, the composition has a viscosity of less than 800,000 centipoise (cP), less than 750,000 cP, less than 200,000 cP, less than 150,000 cP, less than 100,000 cP, less than 90,000 cP, less than 70,000 cP, 50,000 cP, less than 30,000 cP, less than 10,000 cP, less than 1000 cP, less than 500 cP, less than 100 cP, less than 50 cP, less than 10 cP, less than 5 cP, less 1 cP, less than 0.1 cP. In embodiments, the viscosity of the composition increases between 25° C. and 40° C. In some embodiments, the increased viscosity at a higher temperature is about 10,000 cP, about 20,000 cP, about 50,000 cP, about 100,000 cP, about 200,000 cP, about 500,000 cP, about 750,000 cP, about 1,000,000 cP.

In embodiments, the composition comprises a liquid component (e.g., comprising water) and one or more components that form a non-cross linked polymer network, for example, during or after administration to a patient. In some embodiments, the composition has between 50-99% water content providing physical similarity to the targeted tissues. In some embodiments, the composition may be a gel or a composition which forms a gel (e.g., a hydrogel). In some embodiments, the non-cross linked foams have the properties of cross-linked foam products such as non-abrasion and soft touch.

In some embodiments, the composition comprises a liquid component (e.g., comprising water) and one or more components that form a cross-linkable polymer network, for example, during or after administration to a patient. The cross-linking of polymers may occur through chemical means such as initiation of polymerization either by contact as in cyanoacrylates or by external stimuli such as photo-initiation. Gel formation can also be achieved with low molecular weight cross-linkers such as glutaraldehyde or carbodiimide. In another embodiment, the cross-linking may be achieved by using a physical approach such as self-assembling peptides. The composition may comprise one or more biological components such as mussel glue, fibrin tissue adhesives, factor XII, calcium-independent microbial transglutaminase (mTG), and/or gelatin for cross-linking the polymers.

In embodiments, the foam composition is a dissolved gas foam in which the bubble-forming component is a gas or mixture of gasses that are dissolved in the liquid component when under pressure, but form gas bubbles when exposed to ambient pressure. In some embodiments, the dissolved gas foam may comprise a single gas such as nitric oxide, carbon dioxide or oxygen, or a mixture of gases. The gas present in the foam is preferably uniformly dispersed. The gases in the composition may be present as microbubbles, i.e., with small microscopic bubbles not visible to the naked eye. As used herein, the term foam encompasses foams with bubbles of all sizes, including micro foams.

In some aspects, the foam composition comprises a bubble-forming component that comprises a volatile liquid that remains a liquid under pressure, but forms a gas under ambient pressure and/or ambient temperature or body temperature. The composition may comprise one or more volatile liquids that provide gas at the gas-forming temperature/pressure. These include, for example, fluorinated organic compounds, such as fluorinated, including perfluorinated, hydrocarbons, hydrofluorocarbons and hydrofluoroolefins. Examples of such fluorinated organic compounds include trifluromethane, difluromethane, difluroethane, tetrafluroethane, heptafluroethane, perflurobutane, perflurocyclobutane, perfluropentane, perfluorohexane, perfluroheptane, perflurooctane, perflurocyclopentane, perflurocyclohexane, heaxafluropropane, heptafluropropane. In some embodiments, the volatile liquid may be a propellant such as a haloalkane, including 1,1,1,2-tetrafluoroethane (H134A) and/or a hydrofluoroolefin such as 1,3,3,3-tetrafluoropropene (HFO-1234ze(E)). Other volatile liquids that can be used as the bubble forming component of the foam compositions include sulfur hexafluoride; alkanes, such as propane, butanes and pentanes; cycloalkanes and cycloalkenes, such as cyclobutane, cyclopentene, and cyclohexene; dialkyl ethers, such as dimethyl ether, methyl ethyl ether, and diethyl ether; cycloalkylene ethers, such as furan; ketones, such as acetone and methyl ethyl ketone; and carboxylates, such as formic acid, acetic acid, and propionic acid.

In some aspects, the composition may comprise a chemical foam where the bubble forming component comprises one or more chemicals that react to form a gas.

In some specific embodiments, the composition may comprise one or more therapeutic agents described herein, 10-30% poloxamer in water, 0.1-3% xanthan gum, and/or 1-25% polyethylene glycol (PEG). In some embodiments, the foam composition may comprise one or more therapeutic agents described herein, 70-90% water, 2-10% polysorbate 80, 0.01-1.5% methylparaben, 5-10% propylene glycol, 0.05-5.0% xantham gum, and/or 2.5-50% of H134A. In some other embodiments, the foam composition may comprise one or more therapeutic agents described herein, 60-90% of water, 5-15% of propylene glycol, 0.1-1% of xanthan gum, 0.2-1.5% of Lipocol L-23, 0.2-2% of emulsifying wax, 0.1-1% of cetyl alcohol, 0.01-0.1% of methylparaben, 0.01-0.05% of propylparaben, and/or 5-50% of H134A.

In embodiments, the foam composition comprises one or more therapeutic agents, about 10% to about 30% poloxamer in water, about 0.1% to about 3% xanthan gum, and/or about 1% to about 25% PEG. In some embodiments, the foam composition comprises one or more therapeutic agents, about 70% to about 90% purified water, about 2% to about 10% polysorbate 80, about 0.01% to about 1.5% methylparaben, about 5% to about 10% propylene glycol, about 0.05% to about 5.0% xanthan gum. In some embodiments, the foam composition comprises one or more therapeutic agents, about 5% to about 20% propylene glycol, about 0.1% to about 1% xanthan gum, about 0.2% to about 1.5% Lipocol L-23, about 0.2% to about 2% emulsifying wax, about 0.1% to about 1% cetyl alcohol, about 0.01% to about 0.1% methylparaben, and/or about 0.01% to about 0.05% propylparaben. In embodiments, the foam composition comprises one or more therapeutic agents about 15% propylene glycol, about 0.5% Lipocol L-23, about 0.5% emulsifying wax, about 0.3% cetyl alcohol, about 0.05% methylparaben, about 0.01% propylparaben, and about 0.3% xanthan gum. Percentages of components in the compositions provided herein are in percent weight by weight of the composition (% w/w) unless specified otherwise.

### Therapeutic Agents

In one aspect, the foam composition comprises one or more therapeutic agents for the treatment of a GI infection including anti-infective agents, antibiotic agents, anti-inflammatory agents, anti-sporulation agents, probiotic agents, antivirulence agents, immunotherapy such as antitoxin antibodies, bacteriophage, lysins, secondary bile acids, and fecal microbiota transplantation.

### Anti-Infective Agents

An anti-infective agent is any medicine capable to preventing the spread of, or killing, an infectious organism. There are many types of anti-infective drug including but not limited to antibiotics, antifungals, antivirals, and antiparasitics.

### Antibiotic Agents

In embodiments, the foam compositions described herein comprise one or more antibiotic agents including antibiotic agents selected from penicillins, cephalosporins, macrolides, fluoroquinolones, sulfonamides, tetracyclines, aminoglycosides, beta-lactams, monobactams, penems, carbapenems, and combinations thereof. Antibiotic agents are used to inhibit bacteria and some parasites. Each antibiotic may be effective for certain types of infections, therefore the antibiotic administered is based on the most likely cause of infection or on a determination of the infectious organism. Antibiotics are amongst the most prescribed medications worldwide, which has led to the emergence of antibiotic resistance of some bacterial strains. Antibiotics are also known to kill off the healthy microbiota that exists in a patient, leading to additional GI issues. Allergic reactions and other side effects may occur, and interactions may exist between the antibiotic and other medications being prescribed to the patient. Therefore, although antibiotics are usually the first line of defense against bacterial infections, secondary considerations may be taken into account.

In embodiments, the antibiotic agent or agents include one or more of penicillin, cephalosporin, and antifolate/sulfa antibiotic combinations. In some embodiments, and in particular embodiments for treating CDI, the antibiotic agent or agents include one or more of vancomycin, metronidazole, fidaxomicin, tigecycline, rifaximin, rifalazil, ramoplanin, nitazoxanide, nitroimidazole, and combinations thereof.

In embodiments the antibiotic agent or agents include one or more bacteriocins, such a Thuricin CD, Nisin, lacticin 3147, Actagardine A, Actagardine V15F, NVB302, GE2270 or its derivative LFF571, and combinations thereof.

### Anti-Inflammatory Agents

Anti-inflammatory agents, including steroidal and non-steroidal anti-inflammatories act to block certain substances in the body that cause inflammation. Inflammation occurs in several GI infections therefore anti-inflammatory agents may be used to treat symptoms of GI infections.

### Anti-Sporulation Agents

Anti-sporulation agents, including but not limited to cephamycins, inhibit the sporulation pathway of many spore-forming pathogens such as C. difficile for example. Sporulation of C. difficile and other spore-forming pathogens allows infection to persist within the host as well as dissemination of infection through patient-patient contact.

### Probiotic Agents

Probiotics are non-pathogenic microorganisms that can be used to restore a healthy gut microbiota. They can be used as prophylaxis or to treat various ailments such as allergic diseases, urinary tract infections, respiratory infections and GI infections to name a few. There are many types of probiotics including lactobacilli, bifidobacteria and some yeasts. Each has different mechanisms of action and can be used separately or in combination to achieve the desired clinical effect.

### Antivirulence Agents

In embodiments, active agent or agents that can be administered via a foam composition described herein, or in combination with a foam composition described herein, comprise one or more antivirulence agents. Antivirulence agents are agents that block the production or action of virulence factors produced by the microorganism that assist the microorganism in invasion of the host and/oe evasion of host defenses, as well as causing or contributing to disease. Virulence factors include, for example, endotoxins, exotoxins, capsules, and siderophores. Anti-toxin antibodies may be used to treat infections where the resulting disease is caused, at least in part, by the production of one or more toxins by the microorganism, including, for example, diphtheria, tetanus, and C. difficile.

The pathology of CDI is mediated by large clostridial toxins, toxins A (TcdA) and/or B (TcdB). Inhibitors of TcdA and/or TcdB may be used alone or in combination with other agents disclosed herein. In embodiments, the inhibitor of TcdA and/or TcdB is ebselen. In embodiments, the inhibitor of TcdA and/or TcdB is an immunotherapy, e.g., one or more antibodies that target TcdA and/or TcdB, such as bezlotoxumab and/or actoxumab.

### Phage/Lysins

In embodiments, the active agent or agents that can be administered via a foam composition described herein, or in combination with a foam composition described herein, comprise a bacteriophage that is active against the invading bacteria. In embodiments for treating CDI, the bacteriophage targets C. difficile, including for example, DC2, (CD119, (CD27, and (CD6356.

In embodiments, the active agent or agents that can be administered via a foam composition described herein, or in combination with a foam composition described herein, comprise one or more bacteriophage lytic enzymes, or lysins. Endolysins or Lysins, or endolysins, are molecules produced by bacteriophages to digest the bacterial cell wall. Lysins typically consist of two domains: an N-terminal catalytic domain, which provides lytic activity against the host or target species, and a C-terminal binding domain, which binds to particular cell wall structures. The specificity of a lysin is usually dictated by the binding domain, which recognizes a cell wall feature specific to the bacteria that it targets. In embodiments for treating CDI, the lysin targets C. difficile, such as CD27L and PlyCD.

### Secondary Bile Acids

Primary bile acids are metabolized into secondary bile acids by healthy intestinal flora. A lack of secondary bile salts leads to an increased susceptibility to CDI. Secondary bile acids, in particular ursodeoxycholic acid (UDCA), have been demonstrated to inhibit spore germination and vegetative growth of C. difficile strains.

### Fecal Microbiota Transplantation

Fecal microbiota transplantation (FMT) involves transfer of feces from a healthy donor into a patient suffering from a disease due to the imbalance of the gut microbiota. FMT aids in the treatment of these disorders due to the presence of healthy gut bacteria as well as other metabolites such as bile acids, proteins and bacteriophages. In particular, FMT has been used to successfully treat CDI and is becoming an emerging treatment for several other disorders including Parkinson's disease, obesity, insulin resistance, and fibromyalgia.

### Patient Population

The patient may be a human or another mammal. The human patients are typically suffering from a GI infection. In some embodiments, the patient is suffering from an infection caused by Staphylococcus aureus, Bacillus cereus, Clostridium perfringens, Clostridium botulinum, Escherichia coli (ETEC, EPEC, EHEC, EAEC, EIEC), Salmonella sp., Shigella sp., Campylobacter sp., Yersinia enterocolitica, Clostridioides difficile, Vibrio cholerae, Vibrio parahemolyticus, Listeria monocytogenes, Aeromonas hydrophila, or Plesiomonas sp. In some embodiments, the patient is suffering from an infection caused by Entamoeba histolytica or Trichuris trichiura. In embodiments, the patient is suffering from CDI with adynamic ileus. In some embodiments, the GI infection is localized to the colon. In other embodiments the GI infection is spread throughout the intestines. In addition, the infection may be a primary infection or may be recurrent. In embodiments, the patient is a child, is elderly, has difficulty swallowing, is critically ill, has failed oral therapy, has ileus, has altered GI motility, and/or is nauseas.

### Dosage

An effective amount or a therapeutically effective amount of the composition is an amount sufficient to confer a therapeutic benefit in a patient after administration, for example, to improve in the subject one or more symptoms of the disease. The effective amount may vary depending on the species, age, weight, and/or health of the subject and the nature or severity of the disease. In some cases, multiple doses of the composition are administered to achieve the effective amount for the therapeutic benefit intended. In some embodiments, the amount of therapeutic agent present in a single dose of the foaming compositions described herein is about 10%, about 20%, about 30%, about 40%, about 50%, about 60%, about 70%, about 80%, about 90% less than the amount of therapeutic agent present in a single dose used for systemic administration for treating a similar disease at similar stage.

In embodiments, the composition comprises about 0.05% to about 10% (by weight) of an active agent (e.g., vancomycin). In embodiments, the composition comprises about 0.1% to about 5%, about 1% to about 2.5%, about 0.1%, about 0.5% about 1%, about 2%, about 3%, about 4%, about 5%, about 6%, about 7%, about 8%, about 9%, or about 10% (by weight) of an active agent.

In embodiments, one dose of the composition contains about 5 mg to about 5 g of an active agent (e.g., vancomycin). In embodiments, one dose of the composition contains about 10 mg to about 3 g, about 50 mg to about 2 g, about 100 mg to about 1 g, about 100 mg to about 400 mg, about 200 mg to about 800 mg, about 200 mg to about 300 mg, about 300 mg to about 400 mg, about 300 mg to about 700 mg, or about 400 mg to about 600 mg of an active agent. In embodiments, one dose of the composition contains about 50 mg, about 100 mg, about 200 mg, about 300 mg, about 400 mg, about 500 mg, about 600 mg, about 700 mg, about 800 mg or about 900 mg of an active agent.

In embodiments, where the foam composition comprises vancomycin, a single dose of the foam composition may contain about 100 mg to about 900 mg, about 200 mg to about 800 mg, about 300 mg to about 700 mg, about 400 mg to about 600 mg, or about 450 mg to about 550 mg of vancomycin. In embodiments, one dose of the composition contains about 50 mg, about 100 mg, about 200 mg, about 300 mg, about 400 mg, about 450 mg, about 500 mg, about 550 mg, about 600 mg, about 700 mg, about 800 mg or about 900 mg of vancomycin.

In embodiments, where the foam composition comprises fidaxomicin, a single dose of the foam composition may contain about 500 mg to about 3 g, about 600 mg to about 2.5 g, about 700 mg to about 2.3 g, about 800 mg to about 2.2 g, or about 1 g to about 3 g, about 1.5 g to about 2.5 g, or about 1.8 to about 2.2 g fidaxomicin. In embodiments, one dose of the composition contains about 100 mg, about 200 mg, about 300 mg, about 400 mg, about, about 500 mg, about 600 mg, about 700 mg, about 800 mg, about 900 mg, about 1 g, about 1.5 g, about 1.8 g, about 2 g, about 2.2 g, about 2.5 g, or about 3 g of fidaxomicin.

### Methods of Treatment

In embodiments, the disclosure provides a method for treating GI infections present in the GI tract or intestines of a patient by administering a foam composition comprising one or more GI infection therapeutic agents to the patient in need thereof. The subjects receiving the therapy described herein (e.g., a therapeutically effective amount of a foam composition, or compositions, comprising one or more GI infection therapeutic agents) may experience because of the therapy, a reduction or complete absence of the GI infection. In one aspect, the disclosure provides a method of treating, preventing and/or reducing the severity of a GI infection by administering a therapeutically effective amount of foam composition comprising an GI infection therapeutic agent into a patient's body in need thereof.

As used herein, “treating,” “treat,” and “treatment” refer to reducing, relieving, ameliorating, or alleviating at least one of the symptoms of the disease, infection, or disorder. For example, in respect to treating a GI infection, “treating,” “treat,” “treatment” refers to, for example, reducing symptoms of the infection, reducing or eradicating colonization by the bacteria or parasite, and/or reducing or inhibiting proliferation and spread of the infection.

The terms “prevent,” “prevention and the like refer to acting prior to overt disease, infection or disorder onset, to prevent the disease, infection or disorder from developing or to minimize the extent of the disease, infection or disorder, or slow its course of development. For example, in respect to treating a GI infection, “prevent,” “prevention and the like may refer to stopping recurrence or inhibiting spread of an infection. The term “cure,” “curing” and the like refer to heal, to make well, or to restore to good health or to allow a time without recurrence of disease so that the risk of recurrence is small.

The phrase “therapeutically effective amount” is used herein to mean an amount sufficient to achieve a desired beneficial change of physiology in the subject or to cause an improvement in a clinically significant condition in the subject, for example, by delaying, reducing, minimizing, or mitigating one or more symptoms associated with the disease or disorder.

The foam compositions described herein are administered to treat a GI infection, such as CDI. GI infections can be viral, bacterial or parasitic in nature and can be the result of ingestion of contaminated food or water, or disruption of the healthy microbiota due to illness or medications, e.g., antibiotics. GI infections may be spread throughout the GI tract or localized to part of the intestines, e.g., the colon. CDI is a GI infection caused by the spore-forming, anaerobic, toxin-producing intestinal bacterium, Clostridioides difficile (C. difficile). In a specific aspect, the disclosure provides a method for treating CDI by administering a foam composition comprising a CDI therapeutic agent to the affected location of the GI tract of a patient in need thereof. In embodiments, the method of treating CDI comprises administering the foam composition to the proximal colon, for example, via a tube placed during colonoscopy. In embodiments, the method of treating CDI comprises administering the foam composition to the distal colon. In embodiments, the foam composition is administered to one or more of the cecum, ascending colon, transverse colon, descending colon, sigmoid colon and/or the rectum of the patient. The foam compositions may be administered once or may be administered multiple times, for example, every 6 hours, every 12 hours, once a day, or every other day.

In certain aspects, this disclosure provides methods of treating GI infections by administering a foam composition to the GI tract of a patient in need thereof wherein the foam composition comprises one or more of anti-infective agents, antibiotic agents, anti-inflammatory agents, anti-sporulation agents, probiotic agents, anti-toxin antibodies, secondary bile acids, and fecal microbiota transplantation.

The GI infection treated using the present disclosure may be caused by one or more of a bacteria comprising Staphylococcus aureus, Bacillus cereus, Clostridium perfringens, Clostridium botulinum, Escherichia coli (ETEC, EPEC, EHEC, EAEC, EIEC), Salmonella sp., Shigella sp., Campylobacter sp., Yersinia enterocolitica, Clostridioides difficile, Vibrio cholerae, Vibrio parahemolyticus, Listeria monocytogenes, Aeromonas hydrophila, or Plesiomonas sp., or by a parasite comprising Entamoeba histolytica and Trichuris trichiura. The GI infection may be a primary infection, i.e., the first time the patient has been exposed to and infected by the pathogen, or a recurrent infection whereby the patient either relapses after therapy or is reinfected by the pathogen two or more times. The GI infection may be asymptomatic. The GI infection may also be antibiotic resistant and therefore require alternative treatment strategies. Additionally, the GI infection may be dispersed throughout the entire GI tract or be localized to one or more specific areas comprising the stomach, duodenum, jejunum, ileum, transverse colon, ascending colon, descending colon, rectum and anus.

In certain embodiments, this disclosure provides methods of treating CDI comprising administering a foam composition to the intestines of a patient in need thereof wherein the foam composition comprises one or more of the following agents: anti-infective agents, antibiotic agents, anti-inflammatory agents, anti-sporulation agents, probiotic agents, anti-toxin antibodies, secondary bile acids, and fecal microbiota transplantation. In embodiments, the foam composition to treat CDI comprises one or more antibiotic agents such as vancomycin and/or fidaxomicin.

The CDI treated using the present disclosure may be located anywhere in the intestines, or more specifically in one or more of the stomach, duodenum, jejunum, ileum, cecum, transverse colon, ascending colon, descending colon, rectum, and anus. Additionally, CDI may be antibiotic resistant. In some embodiments, the CDI may be a primary infection or in other embodiments the CDI may be recurrent.

In embodiments, the methods provide a combination therapy to treat CDI in a patient, wherein the CDI is treated by administration to the colon of the patient a foam composition comprising an antibiotic such as vancomycin and/or fidaxomicin in combination with a second therapy, such as the therapeutic agent disclosed herein. In embodiments, the second therapy comprises administration of one or more of the therapeutic agents described herein including anti-infective agents, antibiotic agents, anti-inflammatory agents, anti-sporulation agents, probiotic agents, antivirulence agents, immunotherapy such as anti-toxin antibodies, bacteriophage, lysins, secondary bile acids, and fecal microbiota transplantation. The second therapy may include, for example, the enteral administration of one or more antibiotics disclosed herein including vancomycin and/or fidaxomicin.

The second therapy may be administered before, during, and/or after administration of the foam composition described herein.

### Devices for Delivering the Foam Compositions

The devices for delivery of the foam compositions comprise one or more components selected from a reservoir for the therapeutic agent and/or propellant, a foam generator, regulator, actuator, and patient interface.

In one aspect of the disclosure, the device used for the delivery of the composition comprises one or more containers, e.g., two containers. In some embodiments, the devices may comprise containers comprising (i) a foamable composition with one or more therapeutic agents and (ii) a pressurized gas canister; or (i) a foamable composition and (ii) one or more therapeutic agents and pressurized gas.

In some embodiments, the contents of the two containers are combined prior to delivery to the patient or during delivery to the patient. In some specific embodiments, one container is used to “charge” the other container before administering the final composition in the intestines via the rectum. In some embodiments, the compositions of the containers are combined before being administered into the patient's body. In some embodiments, the compositions of the containers are administered into the patient's body in tandem or subsequent to each other.

In some aspect, the disclosure comprises a composition in a single container comprising one or more therapeutic agents, a liquid component, a foaming agent and bubble forming component such as a dissolved gas or volatile liquid. The therapeutic agent can be added to the device through a valve or port prior to administration. In addition, or alternatively, the gas or the propellant can be added to the device through a valve or port prior to administration.

In some aspects of the present disclosure, the device used for the delivery of the composition is a single-use device. In other aspects, the device used for the delivery of the composition can be used multiple times.

The foam compositions described herein can be administered to the GI tract using the devices described herein that have been adapted for delivery of the foam composition to the GI tract. In some embodiments, the composition may be introduced directly to the target area of treatment in a patient's body using the device. The container outlet of the device may be attached to a catheter such that the composition is delivered from the container to the patient through the catheter. In some embodiments, the container outlet of the device may be attached to an applicator nozzle such that the composition is delivered from the container to the patient through the applicator nozzle.

In some embodiments, prior to administration of the foam composition, the container outlet is attached to a applicator nozzle, catheter or other device comprising a lumen or channel (e.g., an endoscope comprising a lumen) such that the composition is delivered from the container to the patient through the lumen of the applicator nozzle, catheter, or other device comprising a lumen. The catheter may be a thin pliable tube that can be inserted into the intestines via the rectum or other route. In some embodiments, the catheter may be a soft hollow tube, which is passed into the intestines of the patient via the rectum. The catheter used in the present disclosure may be a Macy Catheter, and the catheter tube may, or may not be, a fenestrated catheter tube. The catheter may be a rectal catheter. The length of the tube may be adjusted based on the location in the intestines to be treated.

An applicator nozzle may be of various sizes and lengths. The applicator nozzle may be single use or reusable. The applicator nozzle may be solid or flexible. The applicator nozzle can be used for GI infections located in the lower GI tract and colon. In embodiments, the composition may be introduced to a patient's body using an injector, ejector, aspirator pump, eductor-jet pump or other devices using a Venturi effect (the reduction in fluid pressure that results when a fluid flows through a constricted section of a tube).

The catheter, applicator nozzle, or other device may be used to disperse the treatment throughout the entire GI tract or to one or more specific areas comprising the stomach, duodenum, jejunum, ileum, transverse colon, ascending colon, descending colon, rectum and anus. For the treatment of CDI, the foam composition can be administered into the cecum or proximal colon via a tube placed during colonoscopy. Alternatively, the foam composition can be instilled more distally, and as it self-expands it can cover more of the colon proximally.

### Kits

Kits of present for the administration of the foam compositions described herein can include any combination of agents, compositions, components, reagents, administration devices, mechanisms, or other entities provided herein. For instance, a kit for the administration of the foam compositions may include one or more foam composition, an administration device, and a combination therapy agent. Kits may further include a device to facilitate delivery. Any of the kits provided herein can be included in a container, pack, or dispenser together with instructions for administration.

We claim:

1. A method to treat a gastrointestinal infection in a patient in need thereof by administering a foam composition comprising a liquid component, a foaming agent, and one or more therapeutic agents.

2. A method of claim 1, wherein the gastrointestinal infection is a bacterial infection.

3. A method of claim 1, wherein the gastrointestinal infection is a parasitic infection.

4. The method of claim 1, where the gastrointestinal infection is present in the intestines.

5. The method of claim 1, wherein the gastrointestinal infection is present in the colon.

6. The method of claim 1 or 2, wherein the gastrointestinal infection is Staphylococcus aureus, Bacillus cereus, Clostridium perfringens, Clostridium botulinum, Escherichia coli (ETEC, EPEC, EHEC, EAEC, EIEC), Salmonella sp., Shigella sp., Campylobacter sp., Yersinia enterocolitica, Clostridioides difficile, Vibrio cholerae, Vibrio parahemolyticus, Listeria monocytogenes, Aeromonas hydrophila, or Plesiomonas sp.

7. The method of claim 1 or 3, wherein the gastrointestinal infection is Entamoeba histolytica or Trichuris trichiura.

8. The method of any one of claims 1-7, wherein the gastrointestinal infection is a primary infection.

9. The method of any one of claims 1-7, wherein the gastrointestinal infection is a recurrent infection.

10. The method of any one of claims 1-9, wherein the gastrointestinal infection is antibiotic resistant.

11. The method of any one of claims 1-9, wherein the gastrointestinal infection is asymptomatic.

12. A method for treating Clostridioides difficile infection in a patient in need thereof comprising administering to the colon of the patient a foam composition comprising a liquid component, a foaming agent, and one or more antibiotics; wherein the one or more antibiotics comprises vancomycin and/or fidaxomicin.

13. The method of claim 12, wherein the foam composition is delivered into one or more of the cecum, ascending colon, and/or transverse colon of the patient.

14. The method of claim 12, wherein the foam composition is delivered into one or more of the descending colon, sigmoid colon, and/or rectum of the patient.

15. A foam composition for treating a gastrointestinal infection comprising a liquid component, a foaming agent, a bubble-forming component, and one or more therapeutic agents.

16. The composition of claim 15, wherein the bubble-forming component is a volatile liquid or a compressed gas.

17. The composition of claim 16, wherein the gas is a single gas.

18. The composition of claim 16, wherein where the gas is a mixture of gas.

19. The composition of claim 16, wherein the volatile liquid comprises one or more fluorinated hydrocarbons

20. The composition of claim 19, wherein where the one or more fluorinated hydrocarbons is selected from a trifluromethane, difluromethane, difluroethane, tetrafluroethane, heptafluroethane, perflurobutane, perflurocyclobutane, perfluropentane, perfluorohexane, perfluroheptane, perflurooctane, perflurocyclopentane, perflurocyclohexane, heaxafluropropane, and heptafluropropane.

21. The composition of any one of the claims 15-20 where the volatile gas is a haloalkane.

22. The composition of claim 16, wherein the volatile liquid comprises H134A.

23. The composition of claim 16, wherein the volatile liquid comprises ze1234.

24. The composition of any one of claims 15-23, wherein the therapeutic agent is a gastrointestinal infection therapeutic agent.

25. The composition of any one of claims 15-23, wherein the therapeutic agent is selected from one or more of anti-infective agents, antibiotic agents, anti-inflammatory agents, anti-sporulation agents, probiotic agents, anti-toxin antibodies, secondary bile acids, and fecal microbiota transplantation.

26. The composition of any one of claims 15-23, wherein the therapeutic agent comprises one or more of vancomycin, metrolidazole, fidaxomicin, tigecycline, rifaximin, penicillin, cephalosporin, antifolate/sulfa antibiotic combinations, nitroimidazole, penem, glycopeptide, and monobactam antibiotics.

27. The composition of any one of claims 15-26, where the composition forms a foam after administering to the targeted area in the patient's body.

28. A device for delivery of the composition of any one of claims 15-27 comprising a reservoir for the therapeutic agent and propellant, a foam generator, regulator, actuator, and patient interface.

29. The device of claim 28, wherein the device comprises one or more containers.

30. The device of claim 28, wherein the device comprises two containers.

31. The device of any one of claims 28-30, wherein the device comprises containers comprising (i) a foamable composition with one or more therapeutic agents and (ii) a pressurized gas canister.

32. The device of any one of claims 28-30, wherein the device comprises containers comprising (i) a foamable composition and (ii) one or more therapeutic agents and pressurized gas.

33. The device of any one of claims 28-32, wherein the compositions of the containers are combined prior to delivery to the patient.

34. The device of any one of the claims 28-32, wherein the compositions of the containers are administered in tandem to the patient.

35. The device of claim 28, wherein the device comprises a single container containing the foamable agent with one or more therapeutic agents and dissolved gas.

36. The device of claim 28, wherein the devices comprises a single container containing the foamable agent with one or more therapeutic agents and propellant.

37. The device of any one of claims 28-36, wherein the device is a single-use device.
